(19) United States
(12) Patent Application Publication
(10) Pub. No.: US 2024/0009275 A1
(43) Pub. Date: Jan. 11, 2024

(54) COMBINATION IMMUNOTHERAPY OF IL-15 AND CD40 AGONIST IN CANCER TREATMENT

(71) Applicants: UNIVERSITEIT ANTWERPEN, Antwerpen (BE); UNIVERSITAIR ZIEKENHUIS ANTWERPEN, Edegem (BE)

(72) Inventors: Jonas VAN AUDENAERDE, Antwerpen (BE); Evelien SMITS, Antwerpen (BE); Marc PEETERS, Antwerpen (BE)

(21) Appl. No.: 18/039,874

(22) PCT Filed: Dec. 3, 2021
(86) PCT No.: PCT/EP2021/084266

§ 371 Date: Jun. 1, 2023

(30) Foreign Application Priority Data
Dec. 4, 2020 (EP) 20211868.3

(51) Int. Cl. A61K 38/20; A61K 39/395; C07K 16/28; A61P 35/00

(52) U.S. Cl. CPC A61K 38/2086; A61K 39/3955; C07K 16/2878; A61P 35/00; A61K 2039/505

(57) ABSTRACT

The present invention relates to the field of combination immunotherapy, more in particular to a combination comprising IL-15 and a CD40 agonist for use in the treatment of cancer, such as and not limited to mesothelioma (e.g. pleural, peritoneal, pericardial or testicular mesothelioma).

### FIELD OF THE INVENTION

The present invention relates to the field of combination immunotherapy, more in particular to a combination comprising IL-15 and a CD40 agonist for use in the treatment of cancer, such as and not limited to mesothelioma (e.g. pleural, peritoneal, pericardial or testicular mesothelioma).

### BACKGROUND TO THE INVENTION

Mesothelioma is a type of cancer that develops from the thin layer of tissue that covers many of the internal organs (known as the mesothelium). The most common area affected is the lining of the lungs and chest wall. Less commonly the lining of the abdomen and the sac surrounding the heart or testis may be affected. Signs and symptoms of mesothelioma may include shortness of breath due to fluid around the lung, a swollen abdomen, chest well pain, cough, feeling tired and weight loss.

More than 80% of mesothelioma cases are caused by exposure to asbestos. The greater the exposure, the greater the risk. Other risk factors include genetics and infection with the simian virus 40. Treatment options often include surgery, radiation therapy and chemotherapy.

There are typically 4 types of mesothelioma known, being pleural mesothelioma, peritoneal mesothelioma, pericardial mesothelioma or testicular mesothelioma. Pleural mesothelioma is characterized by affecting the lining (mesothelium) of the lung and is by far the most common type of mesothelioma, i.e. approximately 80-90% of all diagnosed mesothelioma cases are pleural. Peritoneal mesothelioma is characterized by affecting the lining of the abdominal cavity (i.e. the peritoneum), Pericardial mesothelioma is characterized by affecting the lining of the heart (i.e. the sac surrounding the heart), but is very rare (i.e. only about 1% of cases). Yet due to the tumor's proximity to the heart, patient may already experience severe symptoms during the early stages of the disease. In most cases pericardial mesothelioma treatment is palliative and only aimed at reducing the symptoms. Testicular mesothelioma is characterized by affecting the lining of the testis (i.e. the sac surrounding the testis), and is also very rare (i.e. only about 1% of cases). Although patients with this type of mesothelioma often have a better life expectance compared to patients having other types of mesothelioma, testicular mesothelioma has a high rate of recurrence.

Agonistic CD40 molecules such as CD40L (CD40 Ligand) have shown promising results in mice bearing malignant mesothelioma. This led to clinical trials where patients were treated with CD40-activating antibodies in combination with cisplatin and pemetrexed. From this study six partial responses (40%) and 9 stable disease (53%) as best response were observed. However, there remains a big margin to increase the potential of CD40 agonists, possible by combinations with other compounds.

IL-15 has also been shown to have the potential to induce potent antitumor activity in NK cells isolated from malignant pleural effusions and to overcome the inhibitory effect of pleural fluid. IL-15 is a versatile cytokine which stimulates both T cell proliferation and generation of cytotoxic lymphocytes, as well as activation and expansion of natural killer (NK) cells. Furthermore, it has the capability to induce CD8 memory cells, thereby playing a crucial role in maintaining long-lasting immune responses to malignant cells and possible prevention of relapse. All these features render it a highly attractive cancer immunotherapeutic as confirmed by its high rank in the NCI's top 20 immunotherapeutic drugs with the greatest potential for broad usage in cancer therapy.

While both molecules have previously been shown to have potential in the treatment of mesothelioma, we have now surprisingly found that in combination they exhibit additive effects in terms of enhanced anti-tumour effect resulting in profound survival increase and even complete cure of the majority of tumours. Also, a striking dose reduction of CD40 agonist was possible by adding IL-15, thereby also reducing the risk of side-effects. Hence, we found that by combining both, at least one of the components can be used at subtherapeutic doses, yet still obtaining a similar efficacy.

### SUMMARY OF THE INVENTION

The present application is directed to a combination comprising IL-15 and a CD40 agonist for use in the treatment of mesothelioma. In particular, the present invention provides a combination of IL-15 and CD40 agonist for use in the treatment of mesothelioma, wherein at least one of said IL-15 and CD40 agonist are used in a subtherapeutic dose.

In a particular embodiment, said CD40 agonist is administered/used at a dose of from about 20 to about 800 μg per kg body weight, preferably from about 30 to about 600 μg per kg body weight, most preferably from about 40 to about 300 μg per kg body weight.

In another particular embodiment, said IL-15 is administered at a dose of from 0.1 to about 50 μg per kg body weight, preferably from about 0.1 to 20 μg per kg body weight, most preferably from about 0.1 to about 2 μg per kg body weight.

In a further embodiment, said IL-15 is administered intravenously at a dose of less than 0.3 μg per kg body weight. In particular where IL-15 is administered via a bolus IV injection, the dose may be less than 0.3 μg per kg body weight. Alternatively, where IL15 is administered via a continuous IV drip system, the dose may be about or below 2 μg per kg body weight. Alternatively, said IL-15 is administered intradermally or subcutaneously at a dose of less than 2 μg per kg body weight.

In yet a further embodiment, the present invention provides a combination as defined herein, wherein at least one of the following applies:
said CD40 agonist is used at a dose of less than 300 μg per kg body weight;
said IL-15 is administered intravenously at a dose of less than 0.3 μg per kg body weight;
said IL-15 is administered via a bolus injection intravenously at a dose of less than 0.3 μg per kg body weight;
said IL-15 is administered via a continuous drip system intravenously at a dose of about or below 2 μg per kg body weight; or
said IL-15 is administered subcutaneously or intradermally at a dose of less than 2 μg per kg body weight.

In another particular embodiment, the combination is in the form of a pharmaceutical composition.

In another particular embodiment, said CD40 agonist is a CD40 antibody or antigen binding fragment thereof such as selected from: Selicrelumab, APX005M, ChiLob7/4, ADC-1013, SEA-CD40, CDX-1140, SGN-40, ABBV-927.

In another particular embodiment, said CD40 agonist is selected from: CD40L, trivalent and hexavalent molecules of CD40L, HERA-CD40L and MED15083.

In another particular embodiment, said IL-15 and said CD40 agonist are administered simultaneously.

In a further embodiment, said IL-15 and said CD40 agonist are administered intravenously or subcutaneously.

In yet a further embodiment, said mesothelioma is selected from the list comprising pleural mesothelioma, peritoneal mesothelioma, pericardial mesothelioma and testicular mesothelioma.

In particular, the inventors found that said combination comprising IL-15 and a CD40 agonist exhibits enhanced anti-tumour effect resulting in profound survival increase. Moreover, the inventors have surprisingly found that a striking dose reduction of CD40 agonist was possible by adding IL-15.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Treatment regime C57B/6j mice were injected with 0.5.10⁸ AE17 cells subcutaneously. When tumours reached a size of 20-25 mm, mice were randomised and treated with isotype control, IL-15, CD40 agonist or IL-15+CD40 agonist according the treatment scheme (FIG. 1A). Timing of dosing is indicated for IL-15 (2.5 μg) with black arrows and for CD40 agonist or the corresponding isotype with red arrows (25 μg).

FIG. 2: Survival Rate—Mesothelioma In FIG. 2 the survival rate of AE17 mesothelioma bearing mice treated as indicated is illustrated (n=7 for the isotype control and IL-15 group, n=8 for the CD40 agonsit and IL-15+CD40 agonist group). Survival was determined by tumour size reaching 150 mm². Mantel-Cox test. All data represent mean±SEM. trend p<0.10; ns p≥0.05 *p<0.05

FIG. 3: Survival Rate—Breast Cancer Female Balb/c mice were injected with 0.5×10E6 67NR breast cancer cells. All protocols used in this experiment or the same as described for the B16F10 Melanoma model. No statistical differences (p<0.05) were observed between the different treatment groups.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

When describing the compounds of the invention, the terms used are to be construed in accordance with the following definitions, unless a context dictates otherwise.

As used in the specification and the appended claims, the singular forms “a”, “an”, and “the” include plural referents unless the context clearly dictates otherwise. By way of example, “a compound” means one compound or more than one compound.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

The present invention thus relates to a combination comprising IL-15 and a CD40 agonist for use in the treatment of mesothelioma.

In the context of the present invention, the term “mesothelioma” is meant to be a type of cancer that develops from the thin layers covering many of the internal organs, mostly the lining of the lungs and chest wall. Less commonly affected linings are those of the abdomen and the sac surrounding the heart or testis. More than 80% of mesothelioma cases are caused by exposure to asbestos. Other risk factors include genetics and infection with the simian virus 40.

The inventors of the present invention have found that the combination according to the present invention exhibits enhanced anti-tumour effect resulting in profound survival increase. An advantage of the combination in accordance with the present invention is therefore enhanced anti-tumour activity. This activity is mediated by an enlarged infiltration of CD8 T cells and NK cells and at the same time a reduction of T regulatory cells. In the present application is provided translational preclinical data on the combination in accordance with the present invention. Moreover, the inventors have found that by using this specific combination, at least one of the components can be used at subtherapeutic dosages, thereby reducing the risk of side-effects associated with high dosages of said components.

Hence, in a particular embodiment, the present invention provides a combination comprising IL-15 and a CD40 agonist for use in the treatment of mesothelioma; wherein at least one of said IL-15 and CD40 agonist are used in a subtherapeutic dose.

More in particular, the present invention provides a combination comprising IL-15 and a CD40 agonist for use in the treatment of mesothelioma in a mammal such as a human being; wherein at least one of said IL-15 and CD40 agonist are used in a subtherapeutic dose.

The term “IL-15”, as used herein, refers to a cytokine that regulates the activation and proliferation of T cells and natural killer (NK) cells. Other names in the art for IL-15 include I115 and interleukin-15. The IL-15 as used in the present invention may be used as such, or may comprise the use of recombinant forms such as rh IL15. Alternatively, it may also comprise the use of IL-15 agonists and superagonists, such as for example RLI-15, IL15 SA or N-803. N-803, formerly ALT-803, is an IL-15 superagonist mutant and dimeric IL-15RαSushi-Fc fusion protein complex that enhances CD8⁺ T and NK cell expansion and function and exhibits anti-tumor efficacy in preclinical models. IL15 SA combines IL-15 and IL15Ra-Fc. RLI-15 is a fusion protein consisting of the NH2-terminal (amino acids 1-77, sushi+) cytokine-binding domain of IL-15Rα coupled to IL-15 via a 20-amino acid flexible linker. This fusion protein, referred to as protein receptor-linker-IL-15 (RLI) acts as an IL-15 superagonists that has an increased serum half-life and biological activity similar to complexed IL-15/IL-15Rα-Fc.

The term “CD40 agonist”, as used herein, refers to a molecule which specifically binds to the subject's CD40 molecule and increases or enhances or induces one or more CD40 activities when it comes in contact with a cell, tissue or organism of the subject expressing CD40.

Anti-CD40 monoclonal antibodies (mAbs) that promote or inhibit receptor function hold promise as therapeutics for cancer and autoimmunity. Rules governing their diverse range of functions, however, are lacking. In this regard, Yu X. et al., 2018 determined characteristics of nine hCD40 mAbs engaging epitopes throughout the CD40 extracellular region expressed as varying isotypes (Yu et al., 2018—Complex Interplay between Epitope Specificity and Isotype Dictates the Biological Activity of Anti-human CD40 Antibodies. Cancer Cell. 2018 Apr. 9; 33(4):664-675.e4). All mAb formats were strong agonists when hyper-crosslinked; however, only those binding the membrane-distal cysteine-rich domain 1 (CRD1) retained agonistic activity with physiological Fc gamma receptor crosslinking or as human immunoglobulin G2 isotype; agonistic activity decreased as epitopes drew closer to the membrane. In addition, all CRD2-4 binding mAbs blocked CD40 ligand interaction and were potent antagonists. Thus, the membrane distal CRD1 provides a region of choice for selecting CD40 agonists while CRD2-4 provides antagonistic epitopes. The present invention thus particularly provides molecules capable of binding the membrane distal CRD1 region. More in particular, a specific class of CD40 agonists suitable for use in the context of the present invention are anti-CD40 monoclonal antibodies.

The CD40/CD40L pathway amplifies the qualitative and quantitative nature of T/NK-APC interactions as highlighted by several key biological observations including (1) up-regulation of co-stimulatory CD80 and CD86 molecules on the APCs; (2) increased levels of class I and II MHC molecules; (3) production of pro-inflammatory cytokines including IL-12, IL-23, TNF, and IL-6; (4) CD4 T cell help that aids the differentiation of naive CD8 T cells into cytolytic effector CD8 T cells; (5) in the case of NK cells helps increasing their activation by producing activating cytokines and faciliting transpresentation of cytokines via APC-NK interaction and (6) in the case of B cells, CD40 signals are absolutely critical for antibody class switching and affinity maturation Accordingly, any of these features may be used alone or in combination with others to define/determine the CD40 antagonistic properties of molecules, and thus the usefulness of said compounds within the context of the invention.

As used herein, “CD40” refers to a cell surface glycoprotein that is a member of the tumor necrosis factor receptor family. Other names in the art for CD40 include: TNFRSF5, p50, CDW40 and Bp50.

In accordance with an embodiment of the present invention, said CD40 agonist is an antibody or antigen binding fragment thereof such as selected from: Selicrelumab (also known as RG 7876 or CP-870,893 or R07009789), APX005M, ChiLob7/4, ADC-1013 (also known as JNJ-64457107), SEA-CD40, CDX-1140, SGN-40, ABBV-927. In accordance with an embodiment of the present invention, said CD40 agonist may also be selected from: CD40L, trivalent and hexavalent molecules of CD40L, HERA-CD40L and MED15083.

The term “antibody” as used herein can include whole antibodies, F(ab′)2 fragment, diabody, triabody, tetrabody, bispecific antibody, monomeric antibodies and any antigen binding fragment (i.e., “antigen-binding portion”) or single-chain variable region fragment (scFv), or disulfide-stabilized variable region fragment (dsFv) thereof. Whole antibodies are glycoproteins comprising at least two heavy (H) chains and two light (F) chains inter-connected by disulfide bonds. Each heavy chain is comprised of a heavy chain variable region (abbreviated herein as VH) and a heavy chain constant region.

In all different embodiments of the present invention, the pharmaceutical combination according to the invention can be administered in a therapeutic or subtherapeutic daily dose to a subject, in particular a human subject. In yet another embodiment, at least one of the components of the pharmaceutical combination of the present invention is administered in a subtherapeutic dose to a subject. In another embodiment, at least one of the components of the pharmaceutical combination according to this invention is administered in a therapeutic dose. In still another embodiment, one of the components of the pharmaceutical combination is administered in a subtherapeutic dose, whereas the other component is administered in a therapeutic dose. In another embodiment, all components of the pharmaceutical combination of the present invention are administered in a subtherapeutic dose to the subject.

In the context of the present invention, a subtherapeutic dose of a therapeutic compound is meant to be a dose which is lower than the usual/typical dose of said compound required to obtain a therapeutic effect. Accordingly, in a preferred embodiment, at least one of the components is administered at a dose that is lower than its dose required to obtain a therapeutic effect, when administered alone.

The term “subtherapeutic amount” of a compound means an amount that would be below an accepted therapeutically effective amount. A subtherapeutic amount can be defined as an amount less than the FDA-approved dosage or dosages for a particular disease. Alternatively, taking into account that many drugs are used off-label, a subtherapeutic amount can be defined as an amount less than that typically prescribed by physicians for a particular disease. A sub-therapeutic amount may also take into account such factors as body mass, sex, age, renal or hepatic impairment, and other parameters which may affect the efficaciousness of a given amount of a particular drug. In certain embodiments, a subtherapeutic amount may be 85% of a therapeutically effective amount. In further embodiments, a subtherapeutic amount may be 70% of a therapeutically effective amount. In further embodiments, a subtherapeutic amount may be 60% of a therapeutically effective amount. In further embodiments, a subtherapeutic amount may be 50% of a therapeutically effective amount. In further embodiments, a subtherapeutic amount may be 40% of a therapeutically effective amount. In further embodiments, a subtherapeutic amount may be 30% of a therapeutically effective amount. In further embodiments, it may be even less.

In other words, a subtherapeutic dose is an amount of a compound/component, which when administered to a patient, is not in itself sufficiently effective in the treatment of the claimed disorders. Yet, the combination as claimed herein was found to be sufficiently effective in the treatment of the claimed disorders, even if one or more of the compounds/components were administered at doses which are not sufficiently effective in the treatment of the claimed disorders.

Where in the context of the present invention, reference is made to a particular dose, this is meant to be the dose to be administered on a given day, even where not explicitly stated so in the current application. For example, where it is stated that the CD40 agonist is administered at a dose of about 20 μg per kg body weight, this is meant to be understood as being administered at a dose of about 20 μg per kg body weight per day. This does not mean that during the treatment regime the dose is given every day of the treatment regime, but rather that on each day of administration of the dose, the given dose per kg body weight is as stated herein.

In a preferred embodiment, in the pharmaceutical combination, the CD40 agonist is administered/used in a dose of from about 20 to about 800 μg per kg body weight, preferably from about 30 to about 600 μg per kg body weight, most preferably from about 40 to about 300 μg per kg body weight. Therapeutic doses currently used in clinical trial typically exceed 300 μg per kg body weight. Depending on the type of CD40 agonist, the subtherapeutic dose may vary. For example, clinically used therapeutic doses for Selicrelumab, amount to about 200 μg whilst for another CD40 agonist, APX005M, the therapeutic dose is 300 μg. Therefore, in a preferred embodiment, the CD40 agonist is administered at a subtherapeutic dose of less than 300 μg per kg body weight; such as less than 250 μg per kg body weight, less than 200 μg per kg body weight; less than 150 μg per kg body weight; less than 100 μg per kg body weight.

In another preferred embodiment, in the pharmaceutical combination, the IL-15 or agonist thereof is administered/used in a dose of from about 0.1 to about 50 μg per kg body weight, preferably from about 0.1 to 20 μg per kg body weight, most preferably from about 0.1 to about 0.3 μg per kg body weight. The dose of administration of IL-15 changes in relation to the method of administration. In particular, when IL-15 is administered intravenously, IL-15 is administered typically at a dose of approximately 0.3 μg per kg body weight in a bolus IV injection, whilst when IL-15 is administered subcutaneously, IL-15 is administered typically at a dose up to 2 μg per kg body weight.

Hence in a particular embodiment, said IL-15 is administered intravenously at a dose of less than 0.3 μg per kg body weight, such as about 0.2 or about 0.1 μg per kg body weight. In particular where IL-15 is administered via a bolus IV injection, the dose may be less than 0.3 μg per kg body weight, such as about 0.2 or about 0.1 μg per kg body weight. Alternatively, where IL15 is administered via a continuous IV drip system, the dose may be about or below 2 μg per kg body weight, such as about or below 1.5, about or below 1.0, about or below 0.5 μg per kg body weight. Alternatively, said IL-15 is administered subcutaneously or intradermally at a dose of less than 2 μg per kg body weight; such as about 1.5 or about 1 μg per kg body weight.

Therapeutic doses currently used in bolus IV injections in clinical trial typically exceed 0.3 μg per kg body weight. Therefore, in a preferred embodiment, the IL-15 or agonist thereof is administered at a subtherapeutic dose of less than 0.3 μg per kg body weight; such as less than 0.2 μg per kg body weight, less than 0.1 μg per kg body weight.

Hence, in a specific embodiment the present invention provides a combination as defined herein, wherein at least one of the following applies:
said CD40 agonist is used at a subtherapeutic dose of less than 300 μg per kg body weight;
said IL-15 is administered intravenously at a subtherapeutic dose of less than 0.3 μg per kg body weight;
said IL-15 is administered via a bolus injection intravenously at a dose of less than 0.3 μg per kg body weight;
said IL-15 is administered via a continuous drip system intravenously at a dose of about or below 2 μg per kg body weight; or
said IL-15 is administered intradermally or subcutaneously at a subtherapeutic dose of less than 2 μg per kg body weight.

The inventors have surprisingly found that a striking dose reduction of CD40 agonist was possible by adding IL-15. An advantage of this embodiment is that adverse effects deriving from the use of the CD40 agonist can be reduced. Moreover, given this synergistic effect of both molecules, in a particular embodiment, both components may even be used at subtherapeutic doses.

By “combination comprising IL-15 and a CD40 agonist”, as used herein, it is referred to any form comprising the said IL-15 and said CD40 agonist which can be administered to a subject. As used herein “a combination” refers to any association between two or more items. The association can be spatial or refer to the use of the two or more items for a common purpose. Hence, in the context of the present invention, the combination as provided herein may be a single composition comprising both components. However, a combination in the context of the invention, also refers to the use of 2 separate compositions, each comprising one of the components, which are nevertheless used in association with each other in the claimed treatment. Said association, may include the simultaneous administration of both compositions to the patient, or the separate administration in such a manner that both components are still able to cooperate in the treatment of the intended disorders.

In another particular embodiment of the present invention, the combination is in the form of a pharmaceutical composition. The pharmaceutical compositions in accordance with the present invention can be for use in human or veterinary medicine.

As used herein, a “composition”, refers to any mixture of two or more products or compounds (e.g. agents, modulators, regulators, etc.). It can be a solution, a suspension, liquid, powder or a paste, aqueous or non-aqueous formulations or any combination thereof. In the context of the present invention, the compositions are preferably pharmaceutical compositions, comprising one or more pharmaceutically excipients, carriers, diluents, . . . .

In the context of the present application, the terms “treatment”, “treating”, “treat” and the like refer to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” covers any treatment of a disease in a mammal, in particular a human, and includes: (a) preventing the disease or symptom from occurring in a subject which may be predisposed to the disease or symptoms but has not yet been diagnosed as having it; (b) inhibiting the disease symptoms, i.e. arresting its development; or (c) relieving the disease symptom, i.e. causing regression of the disease or symptom.

It has to be understood that different methods of administration are suitable to administer the combination in accordance with the present invention. Administration of the combination according to the present invention can be performed using standard routes of administration. Non-limiting embodiments include parenteral administration, such as intradermal, intramuscular, subcutaneous, transcutaneous, or mucosal administration, e.g. intranasal, oral, and the like. In one embodiment in accordance with the present invention, the combination comprising said IL-15 and said CD40 agonist are administered intravenously or subcutaneously. The intravenous administration can be done via a bolus injection or via a continuous IV drip system. The route of administration may determine the therapeutic efficacy, as it was found that subcutaneous administration gives better results than IV bolus injection.

In yet a further embodiment, the mesothelioma is selected from the list comprising: pleural mesothelioma (i.e. lining of the lungs), peritoneal mesothelioma (i.e. lining of the abdomen), pericardial mesothelioma (i.e. lining of the heart) or testicular mesothelioma (i.e. lining of the testicles).

In a further aspect, the present invention provides a method for the treatment of mesothelioma in a subject in need thereof, said method comprising administering to said subject, a combination comprising IL-15 and a CD40 agonist.

In a particular embodiment, the present invention provides a method for the treatment of mesothelioma in a subject in need thereof, said method comprising administering to said subject, a combination comprising IL-15 and a CD40 agonist; wherein at least one of said IL-15 and CD40 agonist are used in a subtherapeutic dose.

### EXAMPLES

### Material and Methods

### Animals

Female C57BL/6J mice, age 6-8 weeks, were obtained from Jackson Laboratories. All mice were maintained at the Animal Core Facility at the University of Antwerp. All animal procedures were conducted in accordance with, and approval of, the Animal Ethics Committee of the University of Antwerp under registration number 2020-11. All mice were housed in filter-top cages enriched with houses and nesting material. Mice were checked on a daily base to inspect health and wellbeing. Mice were given a 7 days adaptation period upon arrival before being included in experiments to reduce stress levels.

### Models and the Experimental Design

In this experiment, a mouse mesothelioma cell line, i.e. AE17, was used. This cell line was cultured in RPMI 1640 cell culture medium supplemented with 10% FBS and 10 mM L-Glutamine. The cell line was maintained at 37° C. and 5% CO₂. The cell line was tested on a routine base for mycoplasma contamination. The cell line was not passaged more than 10 times between freeze and thawing and was only used in experiments between passage two and six.

### Tumour Kinetics and Survival

Prior to injection, AE17 cells were harvested using Tryple, washed trice with sterile PBS and put on a 70 μm cell strainer to assure single cell suspension without any contaminants. Next, mice were injected subcutaneously with 0.5.10⁸ AE17 cells suspended in 100 μl sterile PBS at the left abdominal flank. When tumours reached an average size of 20-25 mm, mice were randomised based on tumour size and divided over four different treatment groups (=day 0): 1. Isotype control; 2. IL-15; 3. αCD40; 4. IL-15+αCD40. Mice were given i.p. 2.5 μg IL-15 (NCI) at days 0-3, 6-10 and 13-14. A mouse agonistic CD40 monoclonal antibody (FGK-45) or corresponding isotype (2A3) was administered i.p. at days 0, 3, 7, 10 and 14 at a dosage of 25 μg per mouse.

Tumour size was measured twice a week using a digital calliper (Chicago Brand). Tumour area was calculated using the formula length×width. Mice were sacrificed when a tumour size of 150 mm² was reached or were stated as long-term survivor when they reached day 100 without reaching this endpoint.

### Statistics

Statistical differences in survival were analysed using a Log-rank test. Differences were considered to be significantly different if p<0.5. Graphs were made using GraphPad v8.0 software. All statistical analyses were carried out in SPSS v26.

### Results

The Combination of IL-15 with a CD40 Agonist Results in Decreased Tumour Volume and Increased Survival in a Mesothelioma Tumor Model

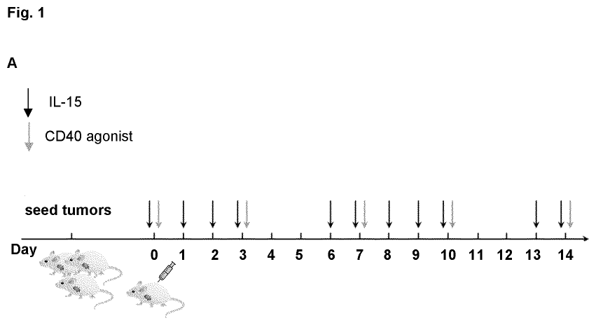

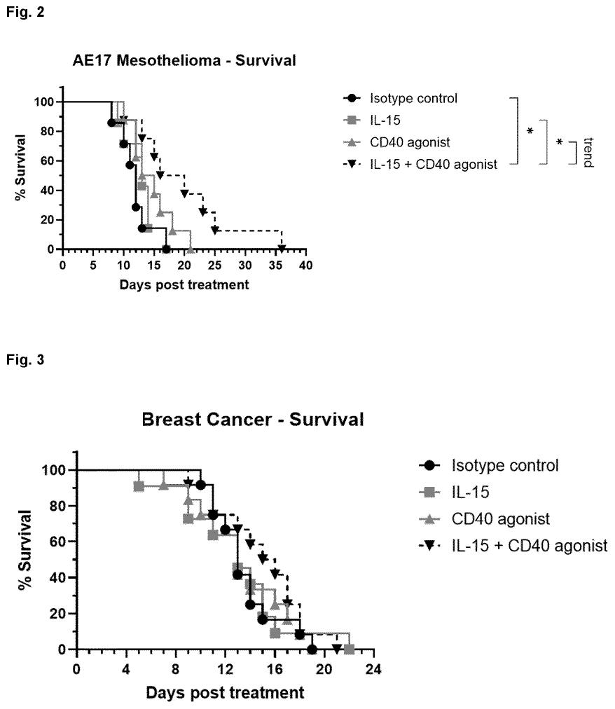

We sought to investigate whether combined treatment of IL-15 and a CD40 agonist can lead to augmented anti-tumour responses in mesothelioma. To investigate this, C57BL/6 mice, bearing AE17 mesothelioma, were treated with IL-15 and/or a CD40 agonist intraperitoneally when tumours reached a size of 20-25 mm (FIG. 1A). We show here that the combination of these agents results in increased survival in this mouse model.

We observed a significant survival benefit between the combination treatment of the isotype control and IL-15 group and a significant trend in survival benefit compared to the CD40 agonist treated group.

There is a significant survival benefit between the combination treatment and the isotype control group (p=0,013) and the IL-15 group (p=0,025). Although there is no significant difference in survival benefit between the combination treatment group and the CD40 agonist monotherapy group, we do observe a significant trend in survival (p=0,089) benefit which will definitely become significant if the sample is increased by pooling a second experimental repeat. Therefore, we provide solid proof that the combination of a subtherapeutic dose of CD40 agonist in combination with IL-15 provides a significant survival benefit in mesothelioma. The above results are data from one experiment with n=7 for isotype control, n=7 for IL-15, n=8 for CD40 agonist and n=8 for IL-15+CD40 agonist group. trend p<0.10; ns p≥0.05 *p<0.05.

The Combination of IL-15 with a CD40 Agonist does not Result in Decreased Tumour Volume and Increased Survival in a Breast Cancer Model

In this example, we assessed whether the beneficial effects of the combined IL15 and CD40 agonist as observed in the melanoma model, could be extended to other tumor models in which mono-therapy of the individual molecules was proposed.

Zippelius et al. (Cancer Immunol Res; 3(3); 236-44. 2015) have demonstrated in a mouse breast cancer model that administration of a CD40 agonist results in prolonged survival compared to untreated mice. Grillgrass et al. (BMC Cancer. 2015 April,—16; 15:293) moreover showed that overexpression of IL-15 promotes tumor destruction in a spontaneous breast cancer model. Accordingly, it was assessed whether the combined use of both molecules would have a similar beneficial effect in the breast cancer model, as observed in the melanoma model.

However, we found on the contrary that when IL-15 and CD40 agonist are combined in a mouse breast cancer model, no survival benefit is observed upon combination of CD40 agonist and IL-15 (FIG. 3), compared to either molecule alone These results demonstrate that the observed beneficial effect of the combination of both molecules is dependent on tumor type and is unpredictable in other tumor models.

1. A combination comprising IL-15 and a CD40 agonist for use in the treatment of mesothelioma.

2. The combination for use as defined in claim 1, wherein at least one of said IL-15 and CD40 agonist are used in a subtherapeutic dose.

3. The combination of claim 2, wherein said subtherapeutic dose of said IL-15 and CD40 agonist is a dose which is lower than the dose of said IL-15 and CD40 agonist required to obtain a therapeutic effect in said mammal, when administered alone.

4. The combination according to anyone of claims 1 to 3, wherein said CD40 agonist is used at a dose of from about 20 to about 800 μg per kg body weight, preferably from about 30 to about 600 μg per kg body weight, most preferably from about 40 to about 300 μg per kg body weight.

5. The combination according to anyone of claims 1 to 4, wherein said IL-15 is used at a dose of from about 0.1 to about 50 μg per kg body weight, preferably from about 0.1 to 20 μg per kg body weight, most preferably from about 0.1 to about 2 μg per kg body weight.

6. The combination according to anyone of claims 1 to 4, wherein said IL-15 is administered intravenously via an IV bolus injection at a dose of less than 0.3 μg per kg body weight; or via a continuous IV drip system at a dose of about or below 2 μg per kg body weight.

7. The combination according to anyone of claims 1 to 6, wherein said IL-15 is administered subcutaneously or intradermally at a dose of less than 2 μg per kg body weight.

8. The combination according to anyone of claims 1 to 7; wherein at least one of the following applies:
said CD40 agonist is used at a dose of less than 300 μg per kg body weight;
said IL-15 is administered via a bolus injection intravenously at a dose of less than 0.3 μg per kg body weight;
said IL-15 is administered via a continuous drip system intravenously at a dose of about or below 2 μg per kg body weight; or
said IL-15 is administered subcutaneously or intradermally at a dose of less than 2 μg per kg body weight.

9. The combination according to anyone of claims 1 to 8, wherein the combination is in the form of a pharmaceutical composition.

10. The combination according to anyone of claims 1 to 9, wherein said CD40 agonist is a CD40 antibody or antigen binding fragment thereof such as selected from: Selicrelumab, APX005M, ChiLob7/4, ADC-1013, SEA-CD40, CDX-1140, SGN-40, ABBV-927.

11. The combination according to anyone of claims 1 to 10, wherein said CD40 agonist is selected from: CD40L trivalent and hexavalent molecules of CD40L, HERA-CD40L and MED15083.

12. The combination according to anyone of claims 1 to 11, wherein said IL-15 and said CD40 agonist are administered simultaneously.

13. The combination according to anyone of claims 1 to 12, wherein said IL-15 and said CD40 agonist are administered intravenously, intradermally or subcutaneously.

14. The combination according to anyone of claims 1 to 13, wherein said mesothelioma is selected from the list comprising: pleural mesothelioma, peritoneal mesothelioma, pericardial mesothelioma and testicular mesothelioma

15. A method for the treatment of mesothelioma in a subject in need thereof, said method comprising administering to said subject, a combination comprising IL-15 and a CD40 agonist.
